(19) United States
(12) Patent Application Publication
(10) Pub. No.: US 2004/0097474 A1
(43) Pub. Date: May 20, 2004

(54) Use of an anti-allergy agent and a steroid to treat nasal conditions

(75) Inventors: Gerald D. Cagle, Fort Worth, TX (US); G. Michael Wall, Fort Worth, TX (US); John M. Yanni, Burleson, TX (US); Rajni Jani, Fort Worth, TX (US); Haresh G. Bhagat, Fort Worth, TX (US)

Correspondence Address: Alcon Research, Ltd., Patrick M. Ryan(Q-148), R&D Counsel, 6201 So. Freeway, Fort Worth, TX, 76134-2099, US

(73) Assignee: Alcon, Inc.

(21) Appl. No.: 10/706,759

(22) Filed: Nov. 12, 2003

Related U.S. Application Data
(60) Provisional application No. 60/425,494, filed on Nov. 12, 2002.

(51) Int. Cl. A61K 31/573; A61L 9/04; A61K 9/14

(52) U.S. Cl. 514/171; 424/46

(57) ABSTRACT

Compositions and methods for treating rhinitis with certain combinations of antiallergic agents and steroids are disclosed.

This application claims priority to U.S. Provisional Application, U.S. Serial No. 60/425,494 filed Nov. 12, 2002.

The present invention is directed to the use of an anti-allergy agent in combination with a steroid to treat nasal conditions, specifically rhinitis.

### BACKGROUND OF THE INVENTION

Allergic rhinitis has historically been treated with a regimen of oral antihistamines and/or oral steroids. Systemic treatment typically requires higher concentrations of the drug compound to be administered to afford an effective concentration to reach the necessary treatment site. Antihistamine compounds are known to have central nervous system (CNS) activity which manifests itself in drowsiness. They may also have anticholinergic activity which manifests itself in the drying of mucus membranes.

Intranasal combination therapy is known. For example, WO 97/01337 discloses combinations of topical nasal antihistamines and topical nasal steroids for the treatment of rhinitis. It does not disclose the use of the combinations of antiallergy agents and steroids of the present invention. WO 97/46243 discloses a nasal spray containing a steroid and an antihistamine. It also does not disclose the combinations of the present invention. There are intranasal products marketed outside the United States that contain both a steroid and an antihistamine, such as: Cortinasal, which contains antazoline and hydrocortisone, from Pharmacobel; Rinosular, which contains diphenhydramine and prednisolone, from SmithKline Beecham; and Rinocusi, which contains diphenhydramine and hydrocortisone, from AlconCusi.

### SUMMARY OF THE INVENTION

The present invention is directed to intranasal compositions containing certain combinations of anti-allergy agents and steroids to treat rhinitis. The anti-allergy agent is selected to be emedastine or olopatadine. The steroid is selected to be fluticasone, mometasone, budesonide or beclomethasone. Methods for the use of the compositions in mammals are also contemplated.

### DESCRIPTION OF PREFERRED EMBODIMENTS

The current invention comprises compositions of either emedastine or olopatadine and a selected steroid for treating the sneezing, rhinorrhea, congestion and itching associated with allergic rhinitis.

Emedastine and olopatadine are known anti-allergy compounds. Emedastine is disclosed in U.S. Pat. No. 4,430,343. Olopatadine is disclosed in U.S. Pat. No. 5,116,863; its use to treat ophthalmic allergic conditions is disclosed in U.S. Pat. No. 5,641,805. The concentration of antiallergy agent in the compositions of the present invention will range from 0.01 to 0.8% (w/v), and is preferably from 0.1-0.8% (w/v) for olopatadine and 0.01-0.1% (w/v) for emedastine. Emedastine is preferably added to the compositions of the present invention in the form of emedastine difumarate. Olopatadine is preferably added in the form of olopatadine hydrochloride.

The combination products of the present invention include a steroid selected from the group consisting of: fluticasone, mometasone, budesonide and beclomethasone. Each of these steroids is known for use in treating rhinitis. The concentration of steroid in the compositions of the present invention will range from 0.01 to 1.0% (w/v), and is preferably 0.02 to 0.5% (w/v). Fluticasone is preferably added to the compositions of the present invention in the form of fluticasone propionate, mometasone as mometasone furoate monohydrate, and beclomethasone as beclomethasone diproprionate. In one embodiment, the steroid is sized using known techniques so that it has an average particle size of 2.5-5 μm. In another embodiment, known nano-sizing techniques are used to obtain steroid particles having an average particle size of less than 0.8 μm, and preferably 0.5 μm or less.

The combinations of the present invention can be incorporated into various types of intranasal formulations for delivery to the nose. For example, the formulations may take the form of solutions or suspensions that are designed to be administered as aerosols, aqueous sprays or drops. Preferably, the formulations are aqueous compositions that are packaged as nasal sprays. The dosing regimen will be set according to the routine discretion of a skilled clinician, but will typically be 1 to 2 sprays of these formulations delivered to the nostrils up to 2 times per day, with each spray delivering 25-100 μL of the formulation.

The formulations may contain, in addition to the anti-allergic agent and the steroid, excipients known in the art of nasal formulations, including antimicrobial agents, antioxidants, agents to increase viscosity, tonicity adjusting agents, buffering agents, solubilizing agents, surfactants, and the like. For example, aqueous intranasal formulations may contain preservatives and preservative adjuncts, such as quaternary ammonium preservatives like benzalkonium chloride and polyquaternium-1, and EDTA; viscosity modifiers, such as hydroxypropyl methylcellulose (HPMC), polyvinyl pyrrolidone, and carboxymethyl cellulose; tonicity adjusting agents, such as sodium chloride, potassium chloride, mannitol, sorbitol, and glycerine; wetting agents/surfactants, such as, tyloxapol or Polysorbate 80; and pH adjusting agents, such as NaOH or HCl. The amount of quaternary ammonium preservative in the formulations of the present invention would typically range from 0.001-0.03% (w/v). The compositions of the present invention are preferably formulated to have a pH of about 3.5 to 8.0 and a viscosity of 1-50 cps.

The following example is illustrative of a composition of the present invention, but is in no way limiting.

### EXAMPLE 1

| Ingredient | % (w/v) |
|---|---|
| Emedastine difumarate | 0.05 |
| Fluticasone propionate | 0.05 |
| Benzalkonium chloride | 0.001-0.03 |
| Disodium EDTA | 0.01 |
| Sodium Chloride (Adjust tonicity to | 0.1 to 0.8 |
| 250-350 mOsmols/kg) | |
| HPMC | 0.1 to 0.5 |
| Tyloxapol | 0.05 |
| Tromethamine | 0.5 |
| NaOH and/or HCl | QS to pH 4-7.7 |
| Purified water | QS to 100 |

### EXAMPLE 2

| Ingredient | % (w/v) |
|---|---|
| Olopatadine | 0.4-0.6 |
| Fluticasone propionate | 0.05 |
| Benzalkonium chloride | 0.001-0.03 |
| Povidone K-29/32 | 1.8 |
| Disodium EDTA | 0.01 |
| Sodium Chloride (Adjust | 0.1 to 0.8 |
| tonicity to 250-350 mOsmols/kg) | |
| Tyloxapol | 0.05 |
| Dibasic sodium phosphate | 0.5 |
| NaOH and/or HCl | QS to pH 4-7.7 |
| Purified water | QS to 100 |

### EXAMPLE 3

| Ingredient | % (w/v) |
|---|---|
| Olopatadine | 0.4-0.8 |
| Fluticasone propionate | 0.05 |
| Benzalkonium chloride | 0.001-0.03 |
| Dibasic sodium phosphate | 0.5 |
| Disodium EDTA | 0.01 |
| Sodium Chloride (Adjust tonicity | 0.6-0.8 |
| to 250-350 mOsmols/kg) | |
| Tyloxapol | 0.05 |
| NaOH and/or HCl | QS to pH 4-7.7 |
| Purified water | QS to 100 |

### EXAMPLE 4

| Ingredient | % (w/v) |
|---|---|
| Olopatadine | 0.4-0.6 |
| Fluticasone propionate | 0.05 |
| Polyquaternium-1 | 0.001-0.03 |
| Povidone K-29/32 | 1.8 |
| Disodium EDTA | 0.01 |
| Mannitol (Adjust tonicity to 250-350 | 0.5-5 |
| mOsmols/kg) | |
| Tyloxapol | 0.05 |
| Boric Acid | 0.5 |
| NaOH and/or HCl | QS to pH 4-7.7 |
| Purified water | QS to 100 |

### EXAMPLE 5

| Ingredient | % (w/v) |
|---|---|
| Olopatadine | 0.4-0.8 |
| Fluticasone propionate | 0.05 |
| Polyquaternium-1 | 0.001-0.03 |
| Dibasic sodium phosphate | 0.5 |
| Disodium EDTA | 0.01 |
| Sodium Chloride (Adjust tonicity | 0.1-0.8 |
| to 250-350 mOsmols/kg) | |
| Boric Acid | 0.5 |
| Tyloxapol | 0.05 |
| NaOH and/or HCl | QS to pH 4-7.7 |
| Purified water | QS to 100 |

### EXAMPLE 6

Olopatadine and Steroid Nasal spray formulations

| | Formulation | | | | | | | | | |
|---|---|---|---|---|---|---|---|---|---|---|
| | 1 | 2 | 3 | 4 | 5 | 6 | 7 | 8 | 9 | 10 |
| Ingredient | % (w/v) | | | | | | | | | |
| Olopatadine hydrochloride | 0.665 | 0.665 | 0.66 | 0.66 | 0.66 | 0.66 | 0.66 | 0.66 | 0.66 | 0.66 |
| | | | 5 | 5 | 5 | 5 | 5 | 5 | 5 | 5 |
| Fluticasone Propionate | 0.05 | 0.05 | 0 | 0 | 0.05 | 0 | 0.05 | 0 | 0.05 | 0 |
| Budesonide | 0 | 0 | 0.03 | 0.03 | 0 | 0.03 | 0 | 0.03 | 0 | 0.03 |
| Povidone | 1.8 | 0.5 | 1.8 | 0.5 | 0 | 0 | 1.0 | 1.0 | 0 | 0 |
| Microcrystaline cellulose | 0 | 0 | 0 | 0 | 0.9 | 0.9 | 0 | 0 | 0.9 | 0.9 |
| Carboxymethyl cellulose | 0 | 0 | 0 | 0 | 0.1 | 0.1 | 0 | 0 | 0.1 | 0.1 |
| sodium | | | | | | | | | | |
| Benzalkonium chloride | 0.001 | 0.001 | 0.001 | 0.001 | 0.001 | 0.001 | 0.001 | 0.001 | 0.001 | 0.001 |
| | — | — | — | — | — | — | — | — | — | — |
| | 0.03 | 0.03 | 0.03 | 0.03 | 0.03 | 0.03 | 0.03 | 0.03 | 0.03 | 0.03 |
| Tyloxapol | 0 | 0 | 0 | 0 | 0 | 0 | 0.05 | 0.05 | 0.05 | 0.05 |
| Polysorbate 80 | 0.005 | 0.005 | 0.005 | 0.005 | 0.005 | 0.005 | 0 | 0 | 0 | 0 |
| Dibasic sodium phosphate, | 0.5 | 0.5 | 0.5 | 0.5 | 0.5 | 0.5 | 0.5 | 0.5 | 0.5 | 0.5 |
| anhydrous | | | | | | | | | | |
| Sodium chloride | q.s. 250- | q.s. 250- | q.s. 250- | q.s. 250- | q.s. 250- | q.s. 250- | q.s. 250- | q.s. 250- | q.s. 250- | q.s. 250- |
| | 350 | 350 | 350 | 350 | 350 | 350 | 350 | 350 | 350 | 350 |
| | mOsm/ | mOsm/ | mOsm/ | mOsm/ | mOsm/ | mOsm/ | mOsm/ | mOsm/ | mOsm/ | mOsm/ |
| | kg | kg | kg | kg | kg | kg | kg | kg | kg | kg |
| Edetate disodium | 0.01 | 0.01 | 0.01 | 0.01 | 0.01 | 0.01 | 0.01 | 0.01 | 0.01 | 0.01 |
| NaOH/HCl | q.s. pH | q.s. | q.s. | q.s. | q.s. | q.s. | q.s. | q.s. | q.s. | q.s. |
| | 4-7.7 | pH 4- | pH 4- | pH 4- | pH4- | pH4- | pH4- | pH4- | pH4- | pH4- |
| | | 7.7 | 7.7 | 7.7 | 7.7 | 7.7 | 7.7 | 7.7 | 7.7 | 7.7 |
| Purified water, QS to | 100 | 100 | 100 | 100 | 100 | 100 | 100 | 100 | 100 | 100 |

We claim:

1. A method for treating allergic rhinitis in mammals which comprises administering a pharmaceutically effective amount of a composition comprising an anti-allergy agent selected from the group consisting of emedastine and olopatadine and a steroid selected from the group consisting of fluticasone, mometasone, budesonide and beclomethasone.

2. The method of claim 1 wherein the amount of anti-allergy agent in the composition is 0.01-0.8% (w/v) and the amount of steroid in the composition is 0.01 to 1.0% (w/v).

3. The method of claim 1 wherein the anti-allergy agent is olopatadine.

4. The method of claim 3 wherein the steroid is fluticasone.

5. The method of claim 1 wherein the steroid has an average particle size of 2.5-5 μm.

6. The method of claim 1 wherein the steroid has an average particle size of less than 0.8 μm.

7. The method of claim 6 wherein the steroid has an average particle size of 0.5 μm or less.

8. The method of claim 1 wherein the composition is an aqueous composition packaged as a nasal spray.

9. The method of claim 1 wherein the composition has a pH of 3.5-8.0 and a viscosity of 1-50 cps.

10. A method for treating allergic rhinitis in mammals which comprises administering a pharmaceutically effective amount of a composition comprising 0.1-0.8% (w/v) of olopatadine and 0.02-0.5% (w/v) of a steroid selected from the group consisting of fluticasone, mometasone, budesonide and beclomethasone, wherein the composition has a pH of 3.5-8.0 and a viscosity of 1-50 cps., and the composition is an aqueous composition packaged as a nasal spray.
